(19) United States
(12) Patent Application Publication
(10) Pub. No.: US 2023/0413734 A1
(43) Pub. Date: Dec. 28, 2023

(54) METHOD OF PRODUCING INTERSPECIFIC HYBRIDS BETWEEN CROSS-INCOMPATIBLE PLANTS

(71) Applicant: UNIVERSITY OF LOUISIANA LAFAYETTE, Lafayette, LA (US)

(72) Inventors: Yi-Hong WANG, Lafayette, LA (US); Chudamani Sharma Prakash, Lafayette, LA (US); Jieqin Li, Lafayette, LA (US)

(73) Assignee: UNIVERSITY OF LOUISIANA LAFAYETTE, Lafayette, LA (US)

(21) Appl. No.: 18/213,638

(22) Filed: Jun. 23, 2023

Related U.S. Application Data
(60) Provisional application No. 63/354,406, filed on Jun. 22, 2022.

(51) Int. Cl. A01G 2/35; A01H 1/02

(52) U.S. Cl. CPC A01G 2/35; A01H 1/02

(57) ABSTRACT

This inventive method comprises regeneration of a hybrid plant via grafting between cross-incompatible species. By grafting and regenerating hybrid from the graft junction in selection media, the graft junction will regenerate hybrid plants with selection for the presence of both species.

### CROSS-REFERENCE TO RELATED APPLICATIONS

This application claims benefit of priority under 35 U.S.C. § 119(e) of U.S. Ser. No. 63/354,406, filed Jun. 22, 2022, the entire contents of which is incorporated herein by reference in its entirety.

### STATEMENT REGARDING FEDERALLY SPONSORED RESEARCH OR DEVELOPMENT

Not Applicable.

### BACKGROUND OF THE INVENTION

Throughout history, plant hybrids have been critical to novel and high-yielding food production. Hybrids produced using the present invention may produce higher yield, are more resistant to plant pathogens and environmental stresses, more nutritious, and more economical or environment-friendly to grow. Even if hybrid weakness does occur, there are ways to obviate the problem such as selection from hybrid progeny populations normal or more desirable plants or producing more hybrids to choose from. After selection and field testing, these hybrids can be used in commercial production.

Grafting is a common horticulture technique that has been in practice for over 2000 years. Use of grafting has been referenced in Bible, ancient Greek, and Chinese text, indicating its practice by at least 5th century BCE (Melnyk and Meyerowitz, 2015). Grafting has been important in agriculture and horticulture for centuries and some of the major benefits from grafting include domestication of woody fruit plants such as apples, pears, and plums, asexual propagation of desirable plants as well as introduction of resistance to various biotic and abiotic stresses and changes in growth habits of scion by altering its characteristic such as size, growth vigor, and fruit yield.

Like sexual hybridization, there is also graft incompatibility between species. In sexual hybridization, due to reproductive barriers, interspecific hybrids between cross-incompatible parents are not viable because of gametic incompatibility (prezygotic) and hybrid breakdown (postzygotic incompatibility).

Similarly, there is also grafting incompatibility, i.e., majority of plants will graft to themselves, fewer will graft to very closely related species, and only rarely will plants successfully graft to more distant relatives.

In eudicot compatible grafting at graft junction, ruptured cells collapse, and the intact cells adhere to the opposing tissue right after grafting. Subsequently, cell divides to give rise to phloem and xylem, followed by the formation of plasmodesmata and cytosolic channels between cells across the junction. In eudicot incompatible grafting, a cell still divides at the junction, but phloem and xylem differentiation may not occur. In both cases, aligning vascular cambia between scions and rootstocks improves grafting success rate.

Mobility of DNA between scions and rootstocks in graft junction has been demonstrated in recent years. For example, it has been demonstrated that plant grafting can result in the exchange of genetic information via either large DNA pieces or entire plastid genomes, complete chloroplast genomes can travel across the graft junction from one species into another and complete nuclear genomes can travel across the graft junction from one species into another. From these studies, graft-induced hybrids were able to be generated. Fuentes et al. (2014) were the first to report hybrid plant (Nicotiana tabauca) that combined nuclear genomes of scions, N. glauca and N. tabacum. These results indicate the transfer of nuclear across the graft junction that produces hybrids.

Grafting to produce new species of plant is a relatively new concept. Although allopolyploidization in plants is common and leads to success of crop domestication as well as speciation and environmental adaptation, cross species allopolyploidization through asexual mechanism is very rare.

In tobacco, the grafting between herbaceous (Nicotiana tabacum) and woody (Nicotiana glauca) tobaccos produced hybrid plant from graft junction which had the genomes from both rootstock and scion and was named Nicotiana tabauca. The allopolypoid hybrid was a result of migration of nuclei from cell to cell at graft junction through plasmodesmata in a cytomixis-like process, not through cell fusion. The movement of entire nuclear genome across the graft junction thus raises the possibility of generating new plant species which has the characters of both parents and might lead to the generation of economically important hybrid plants.

Although grafting in eudicots is common and has been in use for centuries, grafting between monocots is more difficult as monocots have scattered vascular bundles and do not have a vascular cambium. Monocots can be grafted by aligning meristem tissues from scion and rootstock; however, success rates are still be low.

This explains the limited research reported/prior art in the literature concerning monocot grafting. Any prior art focuses exclusively on greenhouse setting grafting. Other prior art had a small, 3-8% graft success rate while grafting monocot plants. Successful grafts generated between para grass (Panicum purpurascens Raddi.) and merker grass (Pennisetum purpureum Schum. var. merkeri) grew and set seeds. However, grafting of monocots in tissue culture setting has not been reported.

Monocots such as maize, rice, and sorghum are some of the more economically important crops. Grafting of such commercially important plants to improve their agronomic traits provides economic benefits. Successful grafting has the potential to provide monocot plants with genetic variation that will improve traits such as growth rate, size, yield, environmental stress tolerances and more.

### SUMMARY OF THE INVENTION

The present invention relates to method to produce interspecific hybrids between cross-incompatible plants through in vitro grafting of scion and rootstock. Scions are shoots or plumules (top half of the embryo) of one species and rootstocks are roots or radicles (the bottom half of the embryo) of another. They are grafted together by removing and switching shoots/plumules from both species using razor blades or biopsy punchers. Live grafts are cultured to regenerate hybrids with a system to select for the presence of both species.

In one embodiment, this invention relates to methods for generating interspecific hybrids between cross-incompatible plants using in vitro or micro-grafting. Two species (1 and 2) each contain a different selection marker such as morphological or resistance to antibiotics or other chemicals are grafted with one as scion and one as rootstock. To graft using embryos, seeds are soaked to initiate germination, plumules in the embryo is removed either by a razor blade, biopsy puncher or any other sharp object. Plumules removed from species 1 switched place with those from species 2 seeds. To graft using young seedlings, seeds are germinated in sterile conditions and grown to young seedlings. For seedlings used as rootstock, shoot meristem is accessed for grafting by a “V” cut or a “T” cut. Scion cut is “V” shaped. The scion is inserted into the rootstock and held together by tubing or sterile clips for two weeks before transferring to regeneration medium. Graft junction will regenerate hybrid plants with selection for the presence of both species.

### BRIEF DESCRIPTION OF THE DRAWINGS

FIG. 1A presents the methods of grafting to regenerate interspecific hybrids with plant embryonic tissues using sorghum and rice as example where plumules are removed (indicated by dash lines) from rice and sorghum by a razor blade, switched, and held with paraffin wax or other sterile materials or objects.

FIG. 1B presents the methods of grafting to regenerate interspecific hybrids with plant embryonic tissues using sorghum and rice as example where plumules are removed by a biopsy puncher and switched between rice and sorghum.

FIG. 2A shows the methods of grafting to regenerate interspecific hybrids with meristem tissues using maize and sorghum.

FIG. 2B describes the methods of grafting to regenerate interspecific hybrids with meristem tissues using sorghum (B) as example.

FIG. 3A shows sorghum-maize hybrid plant one week after transfer to soil.

FIG. 3B shows sorghum-maize hybrid plant at two weeks.

FIG. 3C shows sorghum-maize hybrid plant at four weeks.

FIG. 3D shows sorghum-maize hybrid plant at seven weeks (matured).

### DETAILED DESCRIPTION OF THE INVENTION

The subject matter of the present invention is described with specificity herein to meet statutory requirements. However, the description itself is not intended to necessarily limit the scope of claims. Rather, the claimed subject matter might be embodied in other ways to include different steps or combinations of steps similar to the ones described in this document, in conjunction with other present or future technologies.

The following terms are defined and used herein as follows: Scion: plant part used as shoot in a graft. This can be plumule. Plumule: the rudimentary shoot of an embryo plant that will develop into shoot. Shoot meristem: shoot tip region of potentially indefinite growth and development. Rootstock: a plant part onto which scion is grafted. This can be radicle. Radicle: the bottom part of the embryo that will develop into the primary root. Grafting: freshly cut scion placed onto freshly cut rootstock. The resulting plant combination is called grafts which may or may not need to be held together under sterile conditions for in vitro or micro-grafting. Selection marker: a plant may carry antibiotics or herbicide or other chemical resistance genes through genetic transformations. Alternatively, a plant may have biochemical marker such as (but not limited to) color or metabolic requirement, or morphological marker such as (but not limited to) trichome or stem/leaf hairs. Hybrid: in vitro grafting after regeneration produces hybrids. These can be interspecific and between cross-incompatible parents. These hybrids may possess hybrid vigor (outperform one or both parents) or hybrid weakness (not grow as normal plants). If both parents are diploid, the hybrid may be tetraploids (combining chromosomes from both parents) or aneuploids (chromosome number between tetraploid and diploid as in one of the parents). Hybrids represent novel and advantageous plant varieties important for agricultural or horticultural production. Because the parents are cross-incompatible, the hybrids are asexually produced through in vitro of micro-grafting. Cross-incompatibility: sexually reproducing plants cannot cross with each other or one cannot cross with another and cannot produce fertile progeny containing both parental genomes. Naturally herbicide resistance: existent plants or plants modified through mutation and their progenies may contain genes naturally conferring resistance to certain class(es) of herbicide(s). These plants are resistant to various herbicides and may used in a double selection to produce hybrids which may also be resistant to those herbicides.

Grafting involves the physical connection between shoot of one plant (scion) and the rooted part of another (rootstock) plant. After grafting, plants respond rapidly by activating wound healing to begin the regeneration process. The wound healing process might be activated by the disconnection between leaves and the roots by changing the transport dynamics or by detection of damages to cell in the graft region and subsequent triggering of the plant defense and growth responses.

Graft healing leads to regeneration of tissues around the wound. Normally, grafting creates a compound genetic system by uniting two or more distinct genotypes, each of which maintains its own genetic identity throughout the life of the grafted plant, but the closeness of cells from the two genotypes allows movement of nuclei through plasmadamata in a cytomixis-like process. This produces hybrid cells with chromosomes from both scion and rootstocks and hybrid plants after regeneration. This process can serve as a route to generate asexual allopolyploid hybrids.

The methods according to the present invention can be applied to any plant, preferably higher plants, pertaining to the classes of Angiospermae and Gymnospermae, if they are cross-incompatible. These include plants from the family Acanthaceae, Alliaceae. Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae. Theaceae, and Vitaceae.

In one embodiment, the invention uses embryonic tissues from the seeds of two plants species to be grafted. Each species to be grafted should have a selection marker to identify its genetic or other presence. Such marker may be morphological, biochemical, or genetic. Seeds from each species are soaked under sterile conditions for 2-48 hours to initiate germination process. Plumules from both seeds are removed by razor blades or biopsy puncher or any other sharp object as shown in FIG. 1 using sorghum and rice as an example.

The graft, now with scion on top of the rootstock, is kept under moist and sterile conditions from 5-10 days. Live grafts are then transferred to a culture medium (MS medium supplemented with 3% sucrose). After 1-2 weeks under diurnal cycle of 16 hours light and 8 hours dark at 25° C., growing grafts are transferred to regeneration medium (MS supplemented with 3.5 mg/l BAP, 0.2 mg/l IBA and 0.2 mg/L kinetin) containing selection agent (s) to select for the presence of the parental genomes. The regeneration is under the same growing condition. At this point, selection can be a morphological marker which may or may not express in the hybrid. Regeneration may take 1-4 months. As an embodiment to the invention, hybrids are produced after regeneration. Hybrid plants are grown for evaluation of morphological, horticultural, or agronomical traits.

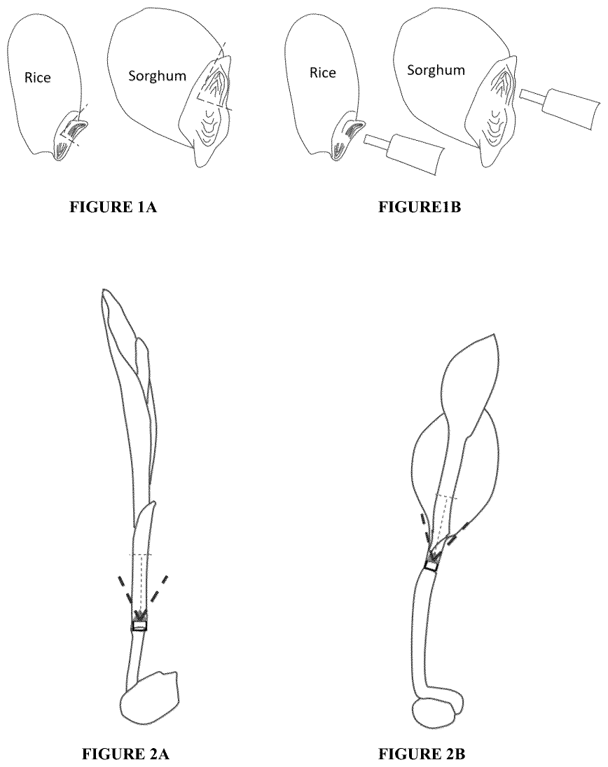

One embodiment of the invention also uses meristem tissues in grafting to produce interspecific hybrid as exemplified using maize and sorghum (FIG. 2). The plant at early seedling stage used as rootstock is cut with either “T” or “V” shaped. The seedling plant used as scion is cut “V” shaped. The scion is inserted into the rootstock and held together by tubing for two weeks before transferring to regeneration medium. The regeneration step is the same as above.

In another embodiment of the invention, if the parental species carry the dominant marker allele and are heterozygous for the dominant allele, the hybrid will be a double heterozygote. If the dominant alleles are represented by A and B, respectively, in the two parental species, then the hybrid's genotype will be AaBb assuming both parents are diploid. Progeny of this hybrid will segregate into 15 (A B, A_bb, aaB_): 1 aabb. If the two parents are originally heterozygous for a single transgene insertion expressed as a dominant allele, then aabb represent a non-transgenic hybrid. If two homozygous non-transgenic resistant to different chemicals are used as the parents, then the hybrids they produce will all be non-transgenic (or non-GMO).

### EXAMPLES

### Example 1

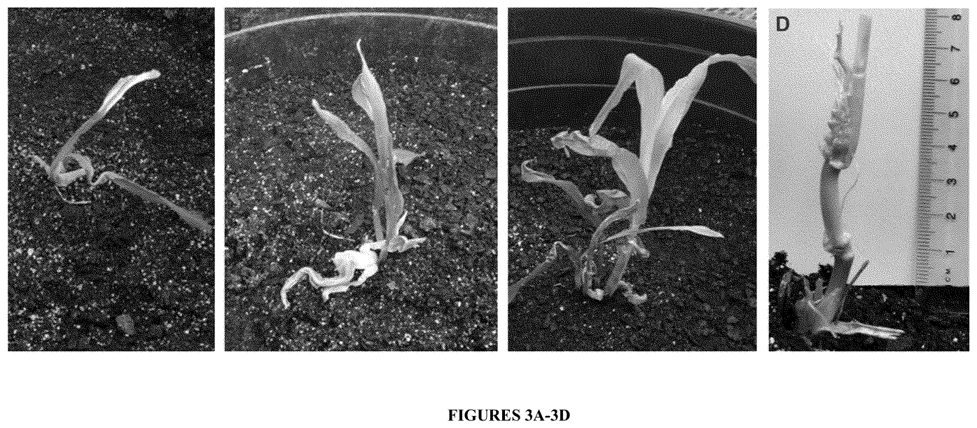

One embodiment of the present invention includes a hybrid plant between sorghum and maize (FIG. 2). The sorghum-maize hybrid looked like sorghum at early stage with smooth green leaves on a thin stem. But growth stalled after three weeks and after another week a new stem started to grow. This time the hybrid plant started to show leaf features of maize plant while the original stem died off. After a month the plant was around 10 cm tall. A seed-bearing ear growing in the stalk toward top of the plant was noticed. This indicated the plant although very small was mature enough to produce a “corn ear” as part of the stalk, not as “ears” on the stalk (FIG. 3).

In one or more embodiments, the invention is as follows: a method of grafting between cross-incompatible plants to regenerate hybrid plants that combine both parental genomes wherein partial parental genomes are combined or wherein full parental genomes are combined. A method of grafting with meristem tissues from young plants as grafting materials with a use of razor blade, biopsy puncher or other sharp objects for interspecific hybrid regeneration wherein said young plants comprise shoots, seedling, and bulbs. A method of grafting with embryonic tissues from soaked or germinating seeds as grafting materials with a use of razor blade, biopsy puncher or other sharp objects for interspecific hybrid regeneration. A method for producing non-transgenic hybrid plants from hybrids generated by the method of claim 1 wherein both parents are genetically transformed or modified. A method for producing non-transgenic hybrid plants from parents naturally resistant to two different herbicides or other chemicals, wherein said hybrid is used in agricultural or horticultural production, in the open field or enclosed environment.

Experiment 2

In another embodiment, surface sterilized transgenic maize (resistant to phosphinothricin) and sorghum (resistant to hygramicin) seeds were grown under aseptic conditions by germinating in Magenta® box with MS Medium supplemented with 3% sucrose. Plants were grown under diurnal cycle of 16 hours light and 8 hours of dark at 25° C.

Grafting was performed using the sterile transgenic plants under aseptic condition in the laminar air flow hood. Stems of similar sized transgenic maize and sorghum plants were cut at approximately 45-degree angle. The scion and rootstock were joined and held together using sterile silicon tubes. The reciprocal grafting was done with each plant serving as both rootstock and scion, giving rise to two grafts. These grafts were grown in MS media supplemented with 3% sucrose and 1 mg/L 2,4-D (2,4-dichlorophenoxyacetic acid) for 2 weeks at 25° C. with 16 hours light and 8 hours dark photo period.

After 2 weeks, the graft site was excised and exposed to regeneration medium containing 50 mg/l hygramicin and 3 mg/l phosphinothricin. The regeneration medium was also supplemented with 3.5 mg/l BAP, 0.2 mg/l IBA and 0.2 mg/L kinetin. Successful selection was defined as the growth of callus followed by production of shoots from the graft region. Plants so produced were transferred to regeneration medium with double selection (phosphinothricin and hygramycin) to produce longer shoot and roots.

Putative hybrid plant was transferred to soil after regeneration of roots in regeneration media followed by 3 days of gentle acclimatization to open air. The plants in soil were transferred to greenhouse and grown at 30° C. with natural daylight with periodic watering and fertilization.

Experiment 2

Over 850 grafts were made between transgenic sorghum and transgenic maize plants in aseptic conditions. Reciprocal cleft grafting was used in majority of grafts; however, in some cases where maize stem size was bigger than sorghum, maize was used as the rootstock and sorghum as scion (FIG. 1).

Silicon tubes pre-sterilized in bleach were used to hold the grafted plants in place and they worked better than paper clips or copper wire and were easy to use.

After 2 weeks of incubation in MS media supplemented with 2,4-D, graft junctions were cut in sterile environments and transferred to selection media. A total of 358 graft junctions (42% of 850) that were properly connected and still living were transferred to selection media.

In the following two weeks the graft junctions were under double selection of hygramycin and phosphinothricin in which 205 of them died. The 153 surviving graft junctions were transferred to regeneration media with double selection. Regeneration media was changed every 3 weeks to supply fresh nutrients and selection.

Following multiple subcultures on regeneration media the number of surviving graft junction with calli was reduced to 30. From these only 6 plantlets (0.59%) regenerated and grew. These six plantlets were transferred to rooting media with double selection, on which 2 more died. Out of remaining four, two were able to produce the roots and thus transferred to soil following acclimatization for 3 days and grown in greenhouse. One plant died after 2 weeks whereas other one started to grow. Two other plantlets in rooting media were subcultured multiple times but died eventually.

The putative hybrid plant looked like sorghum at first: smooth green leaves with a thin stem. But growth stalled after 3 weeks and after another week a new stem started to grow. This time the plant started to show leaf features of maize plant while the original stem died off. After a month the plant was around 10 cm tall. A seed-bearing ear growing in the stalk toward top of the plant was visible. This indicated the plant although very small was mature enough to produce a “corn ear” as part of the stalk, not as “ears” on the stalk (FIG. 2).

In vitro grafting successes of under 1% in this experiment is significant because it shows that the inventive method produces growth from the graft junction between two monocot plants. This implies the transfer of genomic DNA between two plants that produced a new hybrid. This hybrid plant being resistant to both hygramycin and phosphinothricin demonstrates the presence of DNA from both maize and sorghum genomes (FIG. 2).

The hybrid plant had a seed-bearing ear close to the position of maize tassel or sorghum panicle. This may be due to subgenome dominance of maize in the hybrid as demonstrated in allotetraploid Senecio mohavensis in which the allopolyploid hybrids preferentially express genes from one parent with corresponding phenotypic consequences. In the experimental hybrid, the maize genome may have a gene expression bias in its favor.

By producing the hybrid from the graft junction in vitro this experiment demonstrated that different species of monocot plants not only can be grafted but also their nuclear genomes be transferred through graft junction to produce a hybrid. It also showed that the sexually incompatible sorghum and maize are graft-compatible although with the characteristic low success rate of monocots.

Although the invention has been described with reference to the above examples, it will be understood that modifications and variations are encompassed within the spirit and scope of the invention. Accordingly, the invention is limited only by the following claims.

What is claimed is:

1. A method for grafting plants comprising:
a. Identifying a selection marker on a first plant and a second plant, wherein said first plant and said second plant are of difference species, and wherein said first plant and said second plant each comprise: embryonic tissues from seeds of said each of said first plant and said second plant, a rootstock, and a scion;
b. Soaking said seeds of said first plant and said seeds of said second plant under sterile conditions for 2-48 hours to initiate germination;
c. Removing plumules from said seeds of said first plant and removing plumules from said seeds of said second plant;
d. Attaching the scion of said first plant to the rootstock of said second plant to create a first graft and the scion of said second plant to the rootstock of said first plant to create a second graft;
e. Resting said first graft and said second graft under moist and sterile conditions for 5-10 days so that said first graft becomes a live first graft and said second graft becomes a live second graft;
f. Transferring said live first graft and said live second graft to a culture medium and subjecting said transferred live grafts to a diurnal cycle of 16 hours of light and 8 hours of dark at 25° C. to create a growing first graft and a growing second graft; and
g. Transferring said growing first graft and said growing second graft to a regeneration medium comprising selection agents and allowing regeneration for 1-4 months to create a regenerated first graft and a regenerated second graft.

2. The method of claim 1 wherein said marker is selected from the group consisting of: morphological, biochemical, or genetic.

3. The method of claim 1 wherein said removing step is performed by a razor blade or biopsy puncher.

4. The method of claim 1 wherein said first plant is sorghum and said second plant is rice.

5. The method of claim 1 further comprising the step of using either regenerated first graft or regenerated second graft or both to create a hybrid plant.

6. The method of claim 1 wherein said attaching step is performed on meristem tissues.

7. The method of claim 1 wherein said first plant is transgenic maize and said second plant is transgenic sorghum.

8. The method of claim 1 wherein said attaching step is performed by cutting said rootstocks and said scion at a 45-degree angle before attaching.

9. A method for grafting plants comprising:
a. Identifying a selection marker on a first plant and a second plant, wherein said first plant and said second plant are of difference species, and wherein said first plant and said second plant each comprise: embryonic tissues from seeds of said each of said first plant and said second plant, a rootstock, and a scion;
b. Soaking said seeds of said first plant and said seeds of said second plant under sterile conditions for 2-48 hours to initiate germination;
c. Removing plumules from said seeds of said first plant and removing plumules from said seeds of said second plant;
d. Attaching the scion of said first plant to the rootstock of said second plant to create a graft;
e. Resting said graft under moist and sterile conditions so that said graft becomes a live graft;
f. Transferring said live graft to a culture medium and subjecting said transferred live grafts to a diurnal cycle to create a growing graft; and
g. Transferring said growing graft to a regeneration medium comprising selection agents and allowing regeneration to create a regenerated graft.

10. The method of claim 9 further comprising the step of using said regenerated graft to create a hybrid plant.

11. A method of grafting between cross-incompatible plants to regenerate hybrid plants that combine both parental genomes comprising:
a. Soaking seeds of a first plant and the seeds of a said second plant under sterile conditions for 2-48 hours to initiate germination;
b. Removing plumules from said seeds of said first plant and removing plumules from said seeds of said second plant;
c. Attaching the scion of said first plant to the rootstock of said second plant to create a graft;
d. Resting said graft under moist and sterile conditions so that said graft becomes a live graft;
e. Transferring said live graft to a culture medium and subjecting said transferred live grafts to a diurnal cycle to create a growing graft;
f. Transferring said growing graft to a regeneration medium comprising selection agents and allowing regeneration to create a regenerated graft; and
g. Transferring said regenerated graft to rooting media to create a hybrid plant.

12. The method of claim 11 wherein said first plant is transgenic maize and said second plant is transgenic sorghum.
